(19) United States
(12) Patent Application Publication
(10) Pub. No.: US 2024/0138895 A1
(43) Pub. Date: May 2, 2024

(54) NEUROMODULATION OF NASAL NERVES TO TREAT DISEASES

(71) Applicant: Dorna HAKIMIMEHR, Redwood City, CA (US)

(72) Inventor: Dorna HAKIMIMEHR, Redwood City, CA (US)

(21) Appl. No.: 18/050,697

(22) Filed: Oct. 28, 2022

(51) Int. Cl. A61B 18/02

(52) U.S. Cl. CPC A61B 18/02; A61B 2018/00327

(57) ABSTRACT

Neuromodulation of nasal nerves to treat diseases is described where one example of a method for treating dysfunctions or diseases in a patient may generally include introducing a treatment device into a nasal cavity of a patient. The distal end of the treatment device may be navigated into proximity of nerve fibers passing through at least one of an anterior ethmoid foramen, a posterior ethmoid foramen, or a sphenopalatine foramen. The distal end may then be actuated to ablate all or substantially all of the nerve fibers such that the dysfunction or disease is mitigated.

### FIELD OF THE INVENTION

The present invention is related to medical methods and apparatus. More particularly, the present invention is related to methods and devices to treat diseases by modulating the functioning of sensory neurons within the head and neck anatomy.

### BACKGROUND OF THE INVENTION

In addition to the special senses of olfaction, gustation, visual, and vestibular, there is another, sense that is responsive to environmental conditions. Upon activation, this system, can elicit thermal, nociception, or tactile sensations.

This sense often referred to as sensory, general sensory, or common chemical sense is an integral part of the peripheral nervous system, providing somatosensory and vicerosensory information from the organs to the brain. The somatosensory pathway transmits information from the environment to the brain while the visceral sensory component is responsible for monitoring the internal environment within organs to effect and maintain homeostasis. Sensory neurons can respond to different stimuli and can be divided into thermoreceptors, mechanoreceptors, nociceptors, photoreceptors, and chemoreceptors. Visceral sensory nerves transmit pain, stretch, temperature, and chemical change in visceral organs which gets interpreted as sensations like nausea, hunger, gas, cramping, etc. General visceral sensory neurons are considered part of the autonomic nervous system, but unlike the efferent arm, these neurons do not classify as sympathetic or parasympathetic.

For example, the surface of the epithelia of nasal and oral cavities are responsive to tactile and thermal stimuli. Or the peripheral fibers of the trigeminal system, within the ocular, nasal or oral cavities terminating as “free nerve endings”, can be excited by variety of noxious chemicals. This responsiveness to environmental stimuli, is believed to be produced by terminals of thinly or unmyelinated neurons ending in the epithelium, capable of interacting with the environment.

Sensory neurons contain major neuropeptide classes, such as substance P and calcitonin gene-related peptide (CGRP). These neuropeptides are present throughout the peripheral and central nervous system. CGRP is a potent microvascular vasodilator in most vascular beds, especially associated with the heart and trigeminal circulations. Substance P is involved mediating neurogenic vasodilation and increased microvascular permeability. Upon stimulation by environmental or endogenous stimuli, sensory neurons participate in body's reaction to such stimuli by effecting neurogenic influence on the local vasculature and regulating local inflammation, resulting in acute effects such as oedema formation and increased blood flow. The term ‘neurogenic inflammation’ is commonly used to describe the concept that sensory nerves participate in inflammation.

Discovery of transient receptor potential vanilloid 1 (TRPV1) on majority of sensory neurons has provided additional insight in the mechanism by which sensory neurons participate in inflammation. TRPV1 and TRP Ankyrin 1 (TRPA1) channels are activated by distinct chemical agents, with TRPV1 mediating noxious heat and TRPA1 mediating cooler temperatures including noxious cold with effects closely linked to temperature-induced vascular changes. There is evidence that both TRPV1 and TRPA1 have important roles in acute and chronic inflammation. TRP channels (as they are called generally) are implicated in a plethora of physiological processes taking place in the upper airways, including chemosensation, thermosensation, nociception, regulation of the tone and permeability of the vasculature, release of neuropeptides and immune cell mediators, ciliary beating, mucus secretion, and epithelial barrier function.

Sensory neurons have become an important pharmaceutical target in treating several chronic diseases. For example, substance P antagonist is approved for the treatment of nausea and vomiting induced by chemotherapy, CGRP antibodies and antagonists are in late-phase clinical trials for migraine, and TRPA1 antagonists are under clinical trials for diabetic neuropathy. Systemic targeting of sensory neurons comes with significant side effects as these neurons are present in many tissues and organs and the systemic pharmaceutical treatment can not specifically target one diseased tissue, sparing the rest. Therefore, therapies that can target the malfunctioning neurons on a local basis can present a great opportunity in treating multitude of disease states. This is of more importance considering the local interaction between sensory neurons and their free nerve endings and their cellular environment. Local, device-based therapies could offer the potential of treating both the neurons and their cellar neighbors improving the chances for success.

The inventions presented in this application are intended to provide methods and devices to treat diseases by modulating the functioning of sensory neurons within the head and neck anatomy. For example, nasal hypersensitivity can be a direct cause of nasal sensory neuron overreactivity while, in another example, nasal sensory neurons can be stimulated to improve cerebral blood flow in patients suffering from stroke.

Nasal Hyperreactivity

Rhinitis and rhinosinusitis are two chronic upper airway diseases characterized by inflammation of the nasal mucosa that results in pathological features such as rhinorrhea, post-nasal drip, nasal obstruction, nasal itch, sneezing, loss of smell, and/or facial pain and pressure. These symptoms represent exaggerations of normal defensive functions mediated by neural activity or nasal hyperreactivity. Nasal hyperreactivity is defined as “the induction of one or more nasal symptoms like rhinorrhea, sneezing/itch, or obstruction upon encounter of environmental stimuli, such as cigarette smoke, temperature/humidity changes, strong odors/fragrances, and other irritants” (van Gerven et al., 2018).

Nasal mucosa is innervated by three major neural networks: sensory, parasympathetic, and sympathetic. Sensory neurons interact with the environment to detect noxious or potentially harmful agents entering the nasal cavity, acting as the first line of defense in protecting the upper airways. The sympathetic nerve fibers innervate mainly the vascular structures and to a lesser extent the secretory glands, where they release norepinephrine and neuropeptide Y to cause vasoconstriction and to decrease nasal secretion. Parasympathetic fibers innervate both the blood vessels and the glands of the nasal mucosa. Parasympathetic nerve fibers predominantly release acetylcholine and neuropeptide transmitters such as vasoactive intestinal peptide (VIP).

Upon exposure to environmental noxious stimuli, sensory neurons act locally to produce vasodilation, resulting in mucosal swelling to block nasal passages and prevent further influx of the noxious stimuli. Sensory neurons also send signals to the central nervous system (CNS) to activate the effector arm of autonomic nervous system (ANS), i.e., the parasympathetic and/or sympathetic neurons. Parasympathetic neurons cause additional vasodilation and mucus secretion to flush the toxins out while activation of sympathetic arm results in mucus secretion and vasoconstriction. These processes are part of the normal defense system to protect the airways from the ill effects of the environment; however, the delicate balance can be interrupted at various steps of this defense cycle.

Several pathways can result in neural hyperresponsiveness. Hyperreactivity of sensory neurons to environmental stimuli is one of the major causes that can upset the homeostasis of the nasal mucosa. In this scenario, sensory neurons release neuropeptides locally while signaling to the CNS to initiate an effector reflex in response to a normally tolerable and subthreshold stimulus. Increased efficacy of synaptic transmissions in the central nervous system is another pathway through which neural hyperresponsiveness can occur, resulting in an exaggerated effector reflex to a given stimuli. The balance between the sympathetic and parasympathetic response can also result in dysfunctional defense mechanism. For example, rhinitis of the elderly can be traced to dysregulation of the parasympathetic/sympathetic neural balance caused by degeneration of the sympathetic system in favor of the parasympathetic pathway.

In addition to sensory neurons, solitary chemosensory cells (SCCs), present in the nasal mucosa respond to irritants. These cells activate trigeminal CGRP sensitive sensory nerve endings to send signals to CNS and can initiate a neurogenic inflammation similar to that initiated by sensory neurons themselves. This pathway raises the importance of nasal sensory neurons in initiating mucosal inflammatory diseases through multiple avenues.

Nasal Mucosa Innervation

The greater petrosal nerve contains parasympathetic fibers from the facial nerve, and the deep petrosal nerve contains sympathetic fibers from the carotid plexus. The Vidian nerve is formed by the junction of the greater petrosal nerve and deep petrosal nerve and provides preganglionic nerve fibers to pterygopalatine ganglion (also known as sphenopalatine ganglion, SPG). Maxillary nerve, a branch of trigeminal nerve, provides sensory input to SPG. Therefore, pterygopalatine ganglion contains postganglionic parasympathetic and sympathetic fibers along with general sensory fibers of the maxillary nerve. These three types of fibers leave the ganglion as orbital, palatine, nasal and pharyngeal branches.

Nasal branches travel medially from the pterygopalatine ganglion and enter the nasal cavity through the sphenopalatine foramen. These nerve bundles innervate the posterior nasal conchae and posterior lateral nasal wall. The nasopalatine nerve, a branch of palatine nerves passes inferior to the ostium of the sphenoid sinus to reach the septum. It provides sensation to the posteroinferior nasal septum. From here, it courses inferiorly to pierce the hard palate anteriorly, which links the nasal and oral cavities.

The anterior ethmoidal nerve (AEN) is a terminal branch of the nasociliary nerve, a branch of the ophthalmic nerve, itself a branch of the trigeminal nerve. It branches near the medial wall of the orbit. The anterior ethmoidal nerve arises only after the nasociliary has given off its four branches (the ramus communicans to the ciliary ganglion, the long ciliary nerves, the infratrochlear nerve, and the posterior ethmoidal nerve) and provides the nasal mucosa with most of its general sensory innervation.

AEN gives sensory fibers to the meninges and travels through the anterior ethmoidal foramen to reach the anterior cranial fossa. It then moves forward and passes through the cribriform plate to enter the nasal cavity. It gives off branches to the roof of the nasal cavity and bifurcates into a lateral internal nasal branch and medial internal nasal branch and gives sensory fibers to the anterior part of the nasal septum, anterior ethmoid air cells, and the middle ethmoidal air cells. Postganglionic parasympathetic nerve fibers originating from the sphenopalatine ganglion run together with the nasociliary nerve through the ethmoidal foramen and contain VIP, ACh, and nitric synthase (NOS) as their main neurotransmitters (Reuter et al., 1998). AEN is also known to have parasympathetic and some sympathetic content itself (Hosaka et al., 2016).

Nasal Mucosa Blood Supply

The vascular supply of the nasal mucosa involves both external and internal carotid arteries. Five arteries supply the nasal cavity: sphenopalatine artery, anterior and posterior ethmoid arteries, greater palatine artery and superior labial artery.

The sphenopalatine artery (SPA) originates from the maxillary artery and enters the nasal cavity through sphenopalatine foramen. SPA gives rise to two distinct groups, posterior lateral nasal and posterior septal arteries. The posterior lateral nasal branches supply the inferior, middle and superior conchae. The main trunk of SPA then travels along the roof of the nasal cavity to reach the nasal septum. On the septum it gives off branches that run anteriorly on the septum, the most inferior of these branches forms the nasopalatine artery (Osborn', n.d.).

Anterior and posterior ethmoidal arteries originate from the ophthalmic artery, enter the nasal cavity through the cribriform plate and give off branches that anastomose with SPA forming a plexus, mixing the external and internal carotid circulations. The superior labial artery branches from facial artery and supplies the medial wall of the nasal vestibule. The greater palatine artery enters the nasal cavity through the incisive canal and anastomoses with nasopalatine artery (Osborn', n.d.).

All the arteries supplying the nasal mucosa anastomose on anterior inferior wall of the nasal septum, forming a dense plexus. This plexus is referred to as Kisserbach's plexus or little's area. It has been well established that in peripheral tissues, nerves often run along blood vessels, reflecting their need for oxygen and nutrients, as well as their physiological control of vasoconstriction and dilation (Mukouyama et al., 2002). CGPR tracing of nasal mucosa has shown intimate relationship between the blood vessels and sensory neurons. It is therefore reasonable to believe that an arterial plexus, similarly, represents a dense network of interconnected neurons running along these arteries. Strategic location of the little's area at the entrance to the nasal cavity, makes it ideal to access the incoming air and for the sensory neurons to test the incoming air for potential toxins and effect their regulatory function on the mucosal blood vessels.

Nasal Swell Body

In addition to arteries, nasal mucosa blood supply includes capacitance (veins, venules, sinusoids), exchange vessels (capillaries), and arteriovenous anastomosis (AVA). Arteries form a subepithelial network of fenestrated capillaries. Venous sinusoids are interposed between capillaries and venules. Sinusoids are surrounded by smooth muscle and therefore are capable of dilation and constriction. Blood flow into venous sinusoids deep in the mucosa determine the thickness of this tissue, and so the cross-sectional area of airflow through the nose. The nasal mucosal blood flow can respond to a variety of drugs, allergic or inflammatory conditions, changes in activity level or temperature of the inspired air and nasal obstruction (Osborn', n.d.).

Arteriovenous anastomoses carry blood from arteries to the sinusoids. Calcitonin gene related peptide receptors are located on these anastomoses suggesting that this potent vasodilator induces an influx of blood from dilated arteries into sinusoids with engorgement of the sinusoids, thickening of the mucosa, and decreased airway patency. Neuropeptide Y (NPY), a vasoconstrictor, also has receptors on arterioles and arteriovenous anastomoses. NPY is released from a subset of noradrenergic sympathetic neurons. Constriction of the anastomoses and sinusoidal walls leads to deflation of the sinusoids. NPY and norepinephrine may have vasodilator functions on the throttle veins that regulate the flow of blood out of the sinusoids. The loss of blood volume combined with tissue elastic recoil thins the mucosa and increases nasal patency (Baraniuk & Merck, n.d.).

The nasal septal swell body (NSB) is a distinct structure located in the anterior part of the nasal septum adjacent to the anterior part of the middle turbinate and superior part of the inferior turbinate. Nasal septal swell body is covered by a thick, pseudostratified, ciliated epithelium with goblet cells making it well adapted to the drying effects caused by a high airstream impact. NSB has higher blood sinusoids and higher CGPR immunoreactive nerve endings compared to its surrounding tissue confirming its role as part of the expansile vascular tissues in the nasal cavity to regulate air flow (Baraniuk & Merck, n.d.; Meng & Zhu, 2021) similar to that of inferior and middle conchae.

Nasal Hyperreactivity Treatment

Sectioning of the sensory nerves or the parasympathetic efferent arms have been used to treat nasal hyperreactivity. For example, surgical sectioning and energy-based ablation of posterior nasal nerves has been established as treatment for rhinitis (Chang et al., 2020; Hwang et al., 2017). This approach can disconnect sensory, sympathetic, and parasympathetic neural transmission from SPG into posterior part of the nasal cavity to reduce nasal hyperreactivity.

Surgical sectioning and chemical ablation of AEN has also been shown to be effective treatment for rhinitis (Sun, 1990; Wei et al., 2009; Yue, 1995). While sectioning a nerve can provide short term relief, the effect can be short lasting due to the fast rate of nerve regeneration. Additionally, nerve section can only affect one specific nerve, sparing the rest that could well continue the dysfunction. Energy-based ablation techniques provide the advantage of destroying a large section of the nerve and all the additional nerve bundles that feed into a specific region, resulting in complete denervation. Cryoablation of posterior nasal nerve has shown to have long lasting effect in treating rhinitis.

Energy-based denervation of the anterior and/or posterior ethmoid foramen, can disconnect sensory, sympathetic and parasympathetic innervation to the anterior part of the nasal cavity and the anterior parts of the nasal turbinate and as the cerebral arteries. The regions of the nasal mucosa innervated by AEN has the highest exposure to environmental stimuli and contain the highest content of CGPR and substance P. Considering this region to be the point of entry into the nasal and paranasal cavities, and its role as a defensive gate to the upper airway, the high content of its sensory innervation is understandable. Studies investigating the distribution of CGRP neuropeptide concentration in the nasal cavity have interestingly shown the overlap between the areas of the nasal mucosa with highest CGRP density and highest mucosal sensitivity and areas with the most contact with the incoming airflow (Leel et al., 1995; Silverman & Kruger, 1989).

The common areas identified by the above studies include the nasal septum (especially the anterior and mid-section) and anterior portions of the inferior and middle turbinate (Chamanza et al., 2016; Scheibe et al., 2006). The presence of Kisserbach's plexus in the anterior septum mucosa makes it an accessible target for the neuro-vascular interactions.

As described in more detail earlier, nasal septum is innervated anteriorly by anterior ethmoid nerve and posteriorly by nasopalatine nerve, reaching the nasal cavity from the SPG. Nasal septum is exposed to large amount of inhaled airflow, it has a high content of sensory nerve endings to identify environmental irritants and toxins and initiate a defensive reflex e.g., decreasing nasal patency by causing vasodilation which is facilitated by its high venous and vascular network. Posterior nasal septum innervated by nasopalatine nerve innervate significant number of secretory cells and can further reject toxins by mucus production. The exposure of nasal septum to environmental toxins makes it a vulnerable target for mucosal damage and potential dysfunction. Septal mucosa denervation could provide another pathway in treating nasal hyperreactivity, rhinitis or rhinosinusitis. In fact, injection of botulinum toxin into the nasal septum, has shown efficacy in treating rhinitis symptoms (Huang et al., 2022).

Migraine

Pathogenesis of migraine is a matter of ongoing discussion but appears to have close relationships between activation of sensory fibers of the trigeminal nerve resulting in vasodilation and vasoconstriction of the meningeal artery or venous sinuses which to underlie the pain experienced in migraine.

The sensory branches of the trigeminal nerve spread across most of the face and directly innervate much of the cerebral vasculature. Stimulation of these sensory nerves activates a pathway originating at the trigeminal ganglion that leads to release of neurotransmitters, vasodilation, and increase in cerebral blood flow (CBF). CGRP is likely the neurotransmitter that drives this vasodilatory effect. Given the high concentration of CGRP in the trigeminal ganglion, it is likely produced in the ganglion and then transported to the free nerve endings surrounding the cerebral blood vessels, driving in vasodilation, plasma extravasation, trigeminal nerve sensitization and increase in CBF (White et al., 2021).

While release of CGRP increases CBF, previous animal models have demonstrated that inhibition of CGRP does not completely abolish the response, partially due to the trigemino-parasympathetic reflex arc. Stimulation of sensory afferents from the trigeminal nerve results in parasympathetic reflex and further vasodilation of the cerebral vasculature. Parasympathetic nerve fibers have been found to release vasoactive molecules including acetylcholine, vasoactive intestinal peptide (VIP), pituitary adenylate-cyclase-activating polypeptide (PACAP), and nitric oxide (NO), but not CGRP. SPG has gained interest, as the parasympathetic arm of the migraine headache. Repetitive trans-nasal blockade of SPG has shown effectiveness in treating both cluster headache and chronic migraine.

The ophthalmic branch of trigeminal nerve (V1) is a sensory nerve that innervates the upper part of the face, the two thirds of the anterior scalp and anterior part of nasal mucosa. Furthermore, V1 supplies intracranial structures sensitive to pain. Noxious stimuli to the intracranial sensory receptors are transduced predominantly by the ophthalmic branch.

There is close interconnection between the nasal mucosa and migraine headache. Odors are known to be migraine triggers. According to American Headache Society, Osmophobia, a sensitivity to smell, is frequently described in 95% of migraine patients and is known to trigger or worsen attacks. Odorants can activate TRPA-1 peptide on sensory neurons of the nasal mucosa, thereby activating the trigemino-vascular system directly. Odorants stimulate both the olfactory and trigeminal nerve endings in the nasal mucosa as the olfactory neurons and collaterals from the ophthalmic branch of trigeminal nerve (AEN) are intimately coupled in the olfactory mucosa and in the olfactory bulb (Kesserwani, 2021).

Another example of such interconnection is contact point headaches. Mucosal contact point (MCP) is defined as when two mucosal surfaces come in contact with each other. In the nose, MCP is defined by the contact of nasal septal mucosa with mucosa from the lateral nasal wall, which can result from deviated nasal septum touching the inferior turbinate or the lateral nasal wall (Shaikh et al., 2021). The area of contact between the septal mucosa and the mucosa of the lateral nasal wall has been found to have a high concentration of substance P which act as a mediator for the pain perceived as headache or facial pain (Maniaci et al., 2021; Shaikh et al., 2021).

Nasal administration of environmental irritants induces meningeal vasodilatation via a CGRP-dependent mechanism. Trigeminal innervation of both the meninges and nasal epithelium is established, but it is not known whether the two share primary afferent neurons. One hypothesis suggests that nasal irritants excite dural afferent neurons via intraganglionic transmission. In this concept, the irritants would first activate receptors on afferents in the nasal mucosa, leading to subsequent release of neurotransmitters from cell soma in the ganglia and resultant paracrine excitation or sensitization of nearby CGRP containing dural afferent neurons. Another putative mechanism includes transmission of the signal from one peripheral site to another site by axon reflex. It has been shown that nasal and cranial labeled cells are located in close proximity to each other within the TG. Soma of sensory afferents from the face are clustered around cranial arterial afferent soma in the TG, pointing toward the possibility that intraganglionic transmission may have a role. This close proximity suggests the potential that neurotransmitter and/or neuropeptide released from nasal epithelium afferents would have a high likelihood of cross-exciting cranial afferents (Kunkler et al., 2014).

Considering the pivotal role of the ophthalmic branch of trigeminal nerve and especially AEN in affecting the dilation of cerebral blood vessels and its intimate interaction with the olfactory and nasal mucosa, activation of which can cause headache and migraine, it is reasonable to consider AEN as a potential target in treating migraine.

As described earlier Postganglionic parasympathetic nerve fibers originating from the sphenopalatine ganglion run together with AEN through the ethmoidal foramen and contain VIP, ACh, and nitric synthase (NOS) as their main neurotransmitters (Nozaki et al., 2016; Reuter et al., 1998). Therefore, anterior ethmoid foramen is a conduit at which both sensory and parasympathetic fibers to TG and cerebral arteries pass through and therefore provide an additional target for migraine therapy.

Similarly, nasal septum, innervated by both AEN and NPN, has a high density of sensory and parasympathetic neurons innervating both the nasal mucosa and the cerebral arteries. In fact, intra-nasal lidocaine has shown to be effective in managing acute migraine attacks, pointing to the nasal mucosa as a possible target for migraine relief (Chi, Hsieh, Chen, et al., 2019; Chi, Hsieh, Tsai, et al., 2019; Menshawy et al., 2018). High vascularity and innervation by both AEN and NPN makes nasal septum another, easily accessible target for treating migraine.

Olfaction

The olfactory and the trigeminal systems have a close relationship. Most odorants stimulate the sensory neurons of trigeminal system. The olfactory and trigeminal system are functionally connected. It's been shown that trigeminal activation is increased in patients with acquired anosmia. Part of olfactory-trigeminal interaction may occur in the nasal mucosa, where CGRP released from activated trigeminal fibers inhibits the response of olfactory receptors to olfactory stimuli. Trigeminal afferents innervating the nasal mucosa travel through AEN form collaterals innervating the olfactory bulb. There is experimental evidence that these trigeminal endings contribute to inhibitory effects on neurotransmission within the olfactory bulb by CGRP release. Vigorous activation of trigeminal afferents injured by a viral infection such as Covid-19 or other inflammatory processes may contribute to anosmia as well as headache in a similar process as their involvement in causing headaches.

Frequent association of headache and anosmia has been observed in patients with COVID-19 which may originate with inflammatory responses in the nasal mucosa followed by the activation of meningeal nociceptors. CGRP released from activated trigeminal afferents may therefore contribute to suppress the olfactory functions in the nasal mucosa and the olfactory bulb in those patients experiencing simultaneously headache and anosmia.

Cerebrovascular and Neurodegenerative Diseases

The term cerebrovascular disease includes all disorders in which an area of the brain is temporarily or permanently affected by ischemia or bleeding and one or more of the cerebral blood vessels are involved in the pathological process. Restrictions in blood flow may occur from vessel narrowing (stenosis), clot formation (thrombosis), blockage (embolism) or blood vessel rupture (hemorrhage). Lack of sufficient blood flow (ischemia) affects brain tissue and may cause a stroke.

In addition to cerebrovascular diseases and stroke, neurodegenerative diseases such as Alzheimer's or Parkinson's could be exacerbated due to reduced CBF. Alzheimer's disease (AD) is characterized by a decreased regional cerebral blood flow (CBF). It is most likely that a reduction in CBF could displace a pathway leading to AD genesis (Mazza et al., 2011). Similarly, Parkinson's patients show a pattern of reduced CBF (Cheng et al., 2020).

Diving response is referred to physiological changes due to submersion in the water. These physiological reflexes include increased cerebral blood flow, brachycardia, apnea and increased peripheral blood flow resistance (McCulloch et al., 2018). All these reflexes are meant to protect the heart and the brain in anticipation of water submersion and lack of oxygen. Stimulation of AEN has been shown to trigger diving response. In case of AEN section nasopalatine nerve and nasal branches arriving to the nasal cavity form SPG are shown to also increase CBF upon stimulation (Panneton & Gan, 2014, Panneton & Gan, 2014). Sensory neuron stimulation, through a central pathway, results in a parasympathetic reflex response through SPG which results in further vasodilation and increase in CBF in a similar mechanism as described earlier regarding nasal hyperreactivity or in case of migraine.

Considering nasal septum is innervated by both the AEN and nasopalatine nerve, it is reasonable to believe that stimulation of nasal septum could result in increased CBF. Nasopalatine nerve provides sensation to the posteroinferior nasal septum. From here, it courses inferiorly to pierce the hard palate. It is therefore reasonable to assume that intra-oral stimulation of nasopalatine nerve could also result in increase in CBF.

### SUMMARY OF THE INVENTION

Anterior and posterior ethmoid nerves are branches of the nasociliary nerve that enter the nasal cavity through the anterior and posterior ethmoid foramina, respectively. Anterior ethmoid nerve (AEN) enters the superior nasal cavity by descending through the cribriform plate by passing through the nasal slit immediately lateral to the crista galli to supply the mucosa of the anterosuperior half of the nasal septum and lateral nasal wall. It grooves the internal surface of the nasal bone and terminates by piercing nasal cartilage to become the external nasal nerve, supplying the skin of the dorsal nose and the nasal apex. Posterior ethmoid nerve (PEN) supplies the mucosa of the sphenoid and posterior ethmoidal sinuses. Thirty percent of individuals do not have a posterior foramen.

In one embodiment, ablation of AEN with or without simultaneous ablation of PEN is intended to treat nasal hyperreactivity such as that seen in rhinitis and rhinosinusitis.

In another embodiment, ablation of AEN with or without simultaneous ablation of PEN is intended to treat headache such as migraine.

In another embodiment, ablation of AEN with or without simultaneous ablation of PEN is intended to treat olfactory dysfunction such as acquired anosmia caused by trigeminal overreactivity or malfunction e.g., due to viral infection such as COVID-19.

In one embodiment of the present invention ablation of AEN is conducted at the anterior ethmoid foramen. In another embodiment both the AEN and PEN are ablated at their respective foramina.

In another embodiment, AEN is ablated along its course on the nasal septum. In another embodiment, AEN is ablated along its course on the lateral nasal wall. In yet another embodiment, AEN is ablated along its course on both septal and lateral nasal walls.

AEN, PEN, and nasopalatine nerve (NPN), innervate the mucosa of the nasal septum. AEN also innervates the anterior portion of lateral nasal wall. AEN and PEN provide sensory innervation while NPN provides parasympathetic innervation.

In one embodiment, ablation of NPN with or without simultaneous ablation of AEN, and/or PEN is intended to treat nasal hyperreactivity such as that seen in rhinitis and rhinosinusitis.

In another embodiment, ablation of NPN with or without simultaneous ablation of AEN, and/or PEN is intended to treat headache such as migraine.

In one embodiment, ablation of the mucosa of the nasal septum is intended to treat nasal hyperreactivity such as that seen in rhinitis and rhinosinusitis. In another embodiment, ablation of the mucosa of the nasal septum is intended to headache such as migraine.

In another embodiment, ablation of the mucosa of the nasal septum with simultaneous ablation of the AEN on the lateral nasal wall is intended to treat nasal hyperreactivity such as that seen in rhinitis and rhinosinusitis. In another embodiment, ablation of the mucosa of the nasal septum with simultaneous ablation of the AEN on the lateral nasal wall is intended to treat headache such as migraine.

In one embodiment, ablation of septal mucosa includes ablation of the mucosa overlaying the vascular plexus present on the anterior nasal septum (Little's area).

In another embodiment, ablation of septal mucosa includes the ablation of the mid septum and NSB.

In one embodiment the ablation of sensory nerves is achieved using therapeutic agents. In another embodiment the ablation is achieved chemically. In one embodiment the chemical or therapeutic agent is embedded within a matrix and placed against the tissue to be ablated. In one embodiment the matrix includes biodegradable polymers. In another embodiment the matrix includes mucoadhesive agents. In another embodiment the therapeutic or chemical agent is sprayed or otherwise delivered in liquid form to the target tissue.

In one embodiment, the ablation procedures are conducted using an ablation effector device. The device contemplated here comprises a proximal end and distal end connected by an elongate shaft with a central lumen. The proximal end is to be held and manipulated by an operator (e.g., a surgeon) to navigate the distal end into the nasal cavity and further to the anterior and/or posterior ethmoid foramen. The distal end of the device is intended to effect ablation and as such comprises an ablation effector component. The distal end of the device is positioned on the foramen to ablate the nerve.

In another embodiment, the ablation effector is placed along the target nerve's course before ablating. The ablation procedure might have to be repeated to cover the total length of the target nerve and/or its various anatomical locations, e.g., septal, and lateral courses of the nerve. In one example, AEN is ablated by ablating multiple points along its course.

In one embodiment of the above configuration the distal and proximal end of the device are in fluid communication (e.g., liquid and/or gas).

In another embodiment of the device, the distal end of the device is designed to be atraumatic to the nasal mucosa as it navigates through the nasal cavity or when it reaches its target tissue.

The distal end can have a collapsed and an expanded configuration so that during navigation to the target site the distal end is in the collapsed state and is expanded once it reaches the target tissue to conform to the target tissue without compromising its ability to navigate through tight spaces. Upon completion of the ablation procedure the distal end may be collapsed before the device is removed from the nasal cavity.

In another embodiment, the ablation device is a cryogenic device. In this configuration the distal end is placed directly adjacent to target. The temperature of the distal end is then reduced to temperatures in the range of, e.g., −20° C. to −100° C., freezing the adjacent tissue causing neurolysis. The tissue may be kept frozen for a period before the temperature of the distal end is returned to environmental temperature, thawing the tissue at the same time. The freeze and thaw cycle can be carried out multiple times or the device repositioned multiple times to produce the desired effect.

In another embodiment, the device is a cryogenic device that uses the Joules-Thomson (JT) effect to produce the ultra-low temperature. In such a configuration the high-pressure refrigerant gas or liquid is injected through an internal lumen to the distal end of the device such that when the gas leaves the internal lumen at the proximity of the distal end, it expands into an outer lumen causing a drop in temperature. The exhaust gas is then released back to the environment. In another embodiment, the refrigerant gas used to produce the JT effect can also be used to expand the distal end of the device when the distal end is designed to be expandable.

In another embodiment, the refrigerant gas or liquid is directly sprayed on the target tissue. In this embodiment the proximal end of the device, the shaft and the distal end are in fluid communication (e.g., liquid and/or gas). The proximal end of the device is connected to a source of pressurized refrigerant gas or liquid. The device shaft and distal end are designed so that they can navigate through the nasal cavity to reach the target tissue. Once in place, the refrigerant gas or liquid is sprayed on the tissue through the distal end. The distal end is designed to optimize spray pattern and size.

In another embodiment a principle other than the JT system is used to cool the distal end of the device in order to freeze tissue.

In another embodiment the cryogenic device uses nitrous oxide as the refrigerant gas. In another embodiment the cryogenic device uses carbon dioxide as the refrigerant gas. In another embodiment the cryogenic device uses any chlorofluorocarbon, hydrochlorofluorocarbon, hydrofluorocarbon or any mixtures thereof as refrigerant. In yet another embodiment the cryogenic device uses liquid nitrogen as refrigerant.

In one embodiment, the cryogenic device is designed so that the effective freezing radius (the radius of the tissue that reaches temperatures at or below −20° C.) of the device matches the thickness of the tissue to be ablated to prevent damage to the underlying bone.

In another embodiment the device uses radiofrequency energy for ablation. In yet another embodiment the device uses pulsed radiofrequency energy for ablation. In yet another embodiment the device uses microwave energy for ablation. In yet another embodiment the device uses laser for ablation. In yet another embodiment, the device uses ultrasonic and/or high intensity focused ultrasonic energy for ablation.

In another embodiment one point or multiple points of the shaft is malleable so that the angle between the distal end and the shaft can be controlled to allow for the exact positioning of the distal end or to place the distal end in parallel to the target tissue. Such an angle can be controlled in-situ during operation, or the angle can be adjusted according to the patient's anatomy prior to the start of the operation.

In another embodiment the distal end of the device is designed to be malleable.

In one embodiment the ablation effector is designed to have at least one flat or substantially flat surface that can be placed against the nasal septum.

In another embodiment the ablation device operates under endoscopic visualization. In another embodiment the device comprises a visualization component eliminating the need for using another visualization device such as endoscope.

Prior to the ablation procedure the physician might conduct a diagnostic procedure to ensure of the suitability of the ablation target. In some embodiments such diagnostic step might include injecting or applying an anesthetic to the target location.

In one method of use, a patient may be selected for the operation to be treated for nasal hyperreactivity, rhinitis, rhinosinusitis, headache, or olfactory dysfunction, upon consultation with physician. The device is then navigated through the nasal cavity of the patient under endoscopic visualization. Once the distal end of the device is placed in proximity of anterior ethmoid foramen, an ablation mechanism is triggered resulting in ablation of all or substantially all fibers of AEN. Once the ablation of AEN is completed, in some and not all cases, the device is navigated to the posterior ethmoid foramen to ablate all or substantially all nerve fibers passing through the posterior ethmoid foramen. The device is then navigated out of the nasal cavity. In some cases, before navigating the device out of the nasal cavity, the physician might navigate the device towards the sphenopalatine foramen to ablate all or substantially all the fibers of NPN before exiting the nasal cavity.

In another method of use, a patient may be selected for the operation to be treated for nasal hyperreactivity, rhinitis, rhinosinusitis, headache, or olfactory dysfunction, upon consultation with physician. The device is then navigated through the nasal cavity of the patient under endoscopic visualization. Distal, effector, end of the device is placed at a desired location on the course of AEN on the nasal septum, and an ablation mechanism is triggered resulting in ablation of part or substantially all the length of AEN. Once the ablation of AEN on the nasal septum is complete, in some and not necessarily all cases, the device might be placed on the course of AEN on the lateral nasal wall to ablate all or substantially all the length of AEN. The device is then navigated out of the nasal cavity. In some cases, the physician might decide to repeat the ablation procedure on the course of NPN on the nasal septum before navigating the device out of the nasal cavity. Ablation on the course of each nerve might need to be repeated in order to ablate the desired length of the nerve.

In yet another method of use, a patient may be selected for the operation to be treated for nasal hyperreactivity, rhinitis, rhinosinusitis, headache, or olfactory dysfunction, upon consultation with physician. The device is then navigated through the nasal cavity of the patient under endoscopic visualization. The device is then navigated through the nasal cavity and placed on the top of the cavity, where septal and lateral walls of the cavity meet, so that the ablation effector portion of the device is in contact with the course of AEN on both the septal and lateral nasal wall. An ablation procedure is then triggered to ablate part or substantially all the length of AEN before navigating the device out of the nasal cavity. Ablation at multiple points or regions along the course of the nerve might be needed in order to ablate the desired length of the nerve.

In yet another method of use, after patient selection, the device of the current invention in navigated through the nasal cavity and placed in contact with the nasal septum. Ablation is triggered resulting in ablation of substantial area or region of the nasal septum mucosa. In order to ablate substantial surface area of the nasal septum the ablation procedure might be repeated multiple times at the same or different locations. The device is then navigated out of the nasal cavity. In one embodiment ablating the mucosa of the nasal septum includes ablating the mucosa over the Little's area. In another embodiment of this method, ablating mucosa of nasal septum includes ablating the nasal swell body.

As stimulation of the AEN and NPN results in increase in CBF, in one embodiment of the current invention the mucosa of the nasal septum is stimulated in order to increase CBF to treat diseases caused by compromised cerebral blood flow such as stroke. In another embodiment the mucosa of the nasal septum is stimulated in order to increase CBF in patients in whom cerebral blood flow is compromised due to neuro-degenerative diseases such as Parkinson's or Alzheimer's disease.

In one embodiment, stimulating the mucosa of the nasal septum results in stimulating different subtypes of sensory nerve terminals present within the mucosa, e.g., mechanoreceptors, thermoreceptors, or chemoreceptors, or by stimulating solitary chemosensory cells (SCCs). In one embodiment, mucosa is stimulated mechanically, e.g., by a pulsating mechanical pressure. In other embodiments, septal mucosa is stimulated by temperature change, e.g., by application of cold air. In yet another embodiment septal mucosa is chemically stimulated, e.g., by applying bitter tasting chemicals. In another embodiment, septal mucosa is electrically stimulated. In yet another embodiment, septal mucosa is stimulated photonically e.g. by light.

In one embodiment the mucosa over the little's area is stimulated in order to increase CBF.

### BRIEF DESCRIPTION OF THE DRAWINGS

FIGS. 1A-1 and 1A-2 show the schematic sagittal cross sections of the nasal cavity, showing nasal septum 100A and nasal lateral wall 100B.

FIG. 1B shows the schematic of traverse plane of the base of skull, illustrating the cribriform plate 106 and crista galli 111.

FIG. 1C shows the schematic of one example of a device used for treatment.

FIG. 1D shows the device (100C) placed at the anterior ethmoid foramen 103 to ablate nerve fibers passing through this foramen.

FIG. 1E shows the device placed on a portion of the anterior ethmoid nerve 101 on the nasal septum to ablate at least a portion of this nerve.

FIG. 1F shows the device placed on a portion of the anterior ethmoid nerve 101′ on the nasal lateral wall to ablate at least a portion of this nerve.

FIG. 1G shows the device of the current invention, placed on a portion of nasopalatine nerve 102 on the nasal septum to ablate at least a portion of this nerve.

FIG. 1H shows a cross section of the device placed within the nasal cavity such that it touches on both septal and lateral walls of the nasal cavity and as such can ablate at least a portion of anterior ethmoid nerve on both walls at the same time.

FIG. 2 shows the schematic sagittal cross section of nasal septum and blood vessels supplying it. It also shows the Little's area where blood vessels supplying the nasal cavity form a plexus.

FIGS. 3A and 3B show coronal and sagittal cross section of nasal cavity, respectively, pointing to the approximate location of the nasal swell body 304.

FIG. 4A shows a schematic of the device where the elongated shaft of the device is malleable.

FIG. 4B depicts an embodiment of the device where the distal end of the device is malleable.

FIG. 5A shows the cross section of the distal end of the device, depicting an illumination and visualization components included within the distal end.

FIG. 5B shows the cross section of the distal end of the device where it comprises an ablation effector, illumination component and imaging component.

FIG. 5C shows an example of a navigation system being used to accurately guide the distal end of the device within the nasal cavity.

FIG. 6A shows the distal end of the device in its collapsed state.

FIG. 6B shows the distal end of the device in its expanded state.

FIG. 7 shows a flow chart of an example of a method to treat diseases.

### DETAILED DESCRIPTION OF THE INVENTION

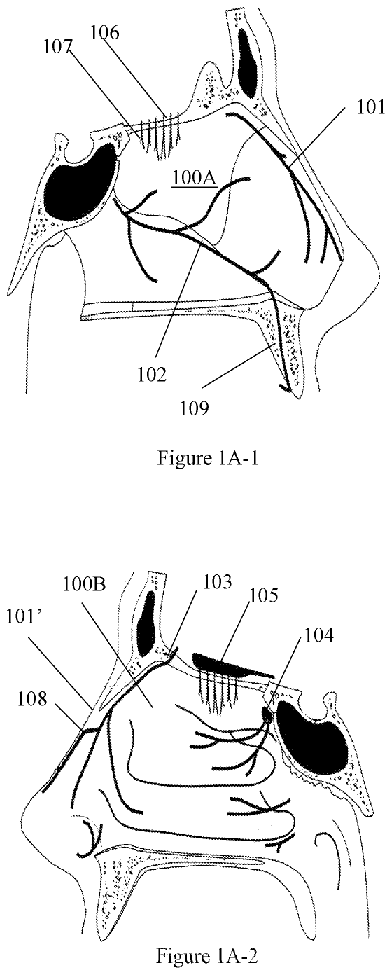

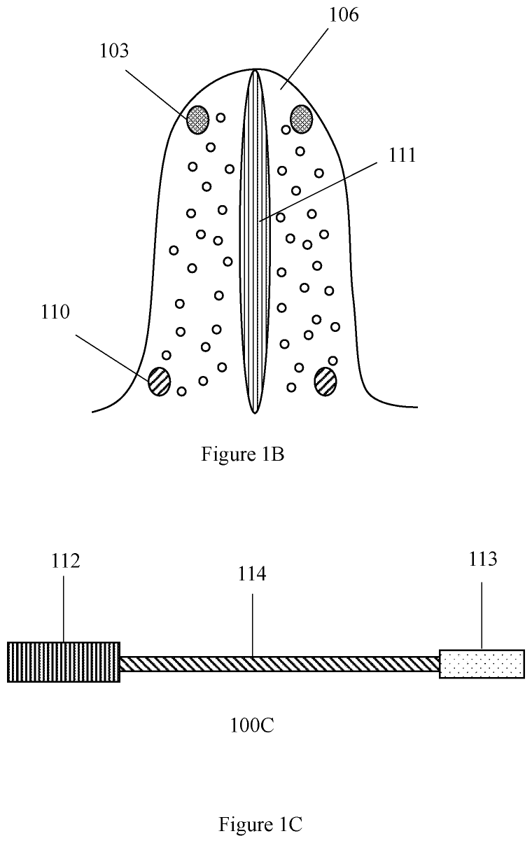

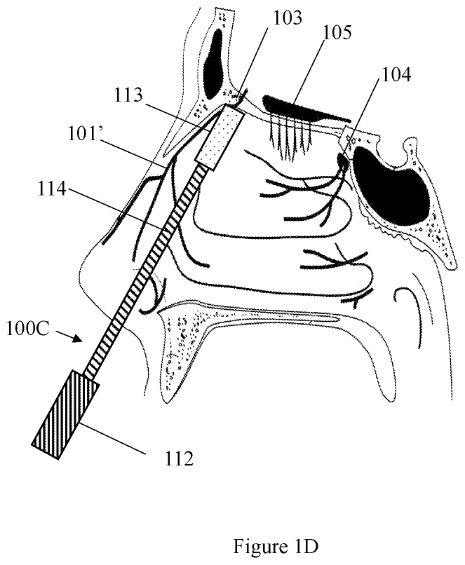

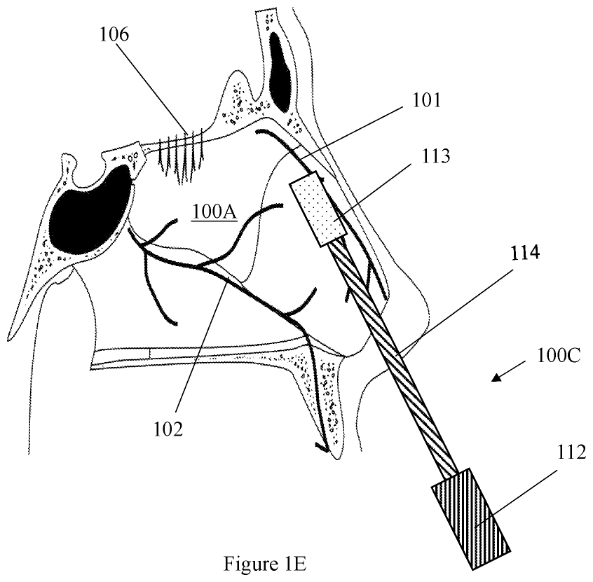

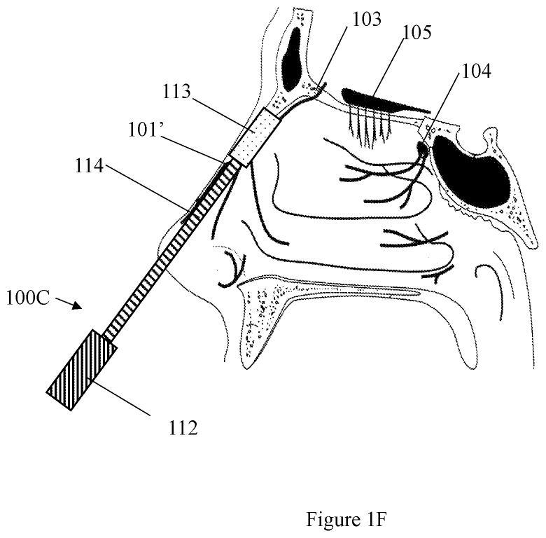

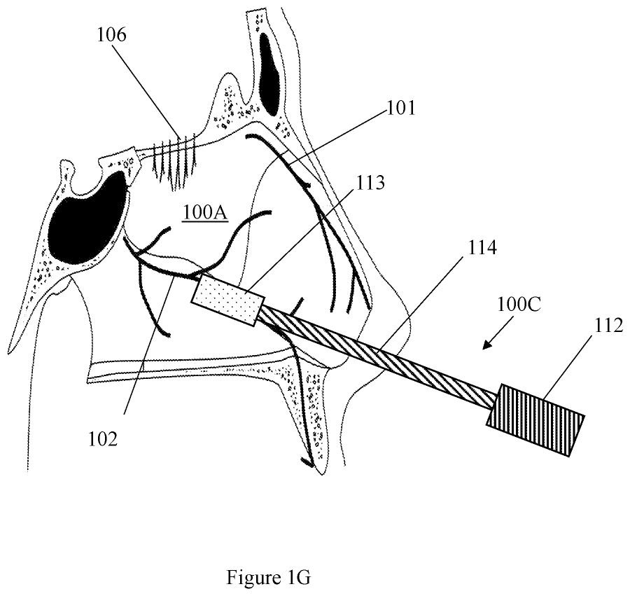

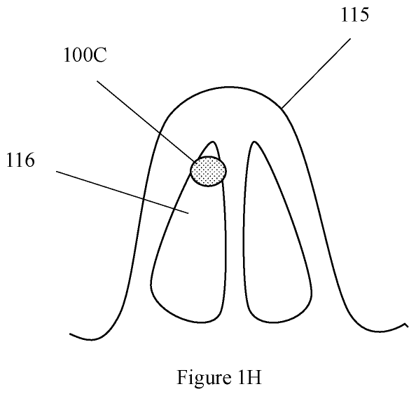

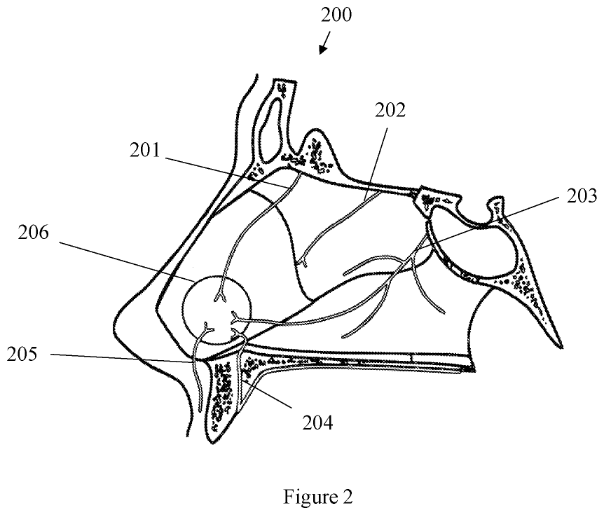

FIGS. 1A-1 and 1A-2 shows side views of the nasal septum 100A, lateral nasal wall 100B, septal AEN 101, lateral AEN 101′, NPN 102, anterior ethmoid foramen 103, sphenopalatine foramen 104, olfactory bulb 105, cribriform plate 106, primary olfactory neurons 107, external nasal nerve 108, and incisive canal 109. Posterior ethmoid nerve and foramen are not shown in this figure. FIG. 1B shows the schematics close up anterior view of cribriform plate 106, anterior ethmoid foramen 103, posterior ethmoid foramen 110, and crista galli 111. FIG. 1C shows a schematic side view of the device 100C with proximal end 112 (e.g., having a handle), distal end 113 having an end effector for applying a treatment stimulation (described in further detail herein), and elongated shaft 114.

In one embodiment, device 100C is navigated into the nasal opening and through the nasal cavity to the proximity of cribriform plate 106 under endoscopic visualization, as schematically depicted in FIG. 1D. Distal end 113 is placed in proximity of anterior ethmoid foramen 103 and an ablation mechanism is triggered to ablate all or substantially all nerve fibers passing through this foramen.

In this and in each of the various embodiments describing how the device may be used to treat the various nerves or portions of the nerves, treatment may be effected for ablating or stimulating the specific nerves for the treatment of the various conditions described herein.

In another embodiment, device 100C is navigated to position its distal end effector 113 at or near the posterior ethmoid foramen 110 (depicted in FIG. 1B) and an ablation mechanism is triggered to ablate all or substantially all nerve fibers passing through this foramen.

In yet another embodiment, device 100C is navigated to sphenopalatine foramen 104 to ablate all or substantially all nerve fibers passing through this foramen.

In one embodiment both anterior ethmoid foramen 103 and posterior ethmoid foramen 110 are ablated. In another embodiment both anterior ethmoid foramen 103 and sphenopalatine foramen 104 are ablated. In yet another embodiment anterior ethmoid foramen 103 and posterior ethmoid foramen 110 and sphenopalatine foramen 104 are ablated.

In one embodiment of the current invention device 100C is navigated through the nasal cavity. Distal end 113 is placed against the mucosal tissue of the nasal septum 100A so that it is in contact with at least a portion of AEN 101, as depicted schematically in FIG. 1E. An ablation mechanism is triggered and at least a portion of AEN 101 is ablated. If desired, the ablation can be repeated so that all or substantially all of AEN 101 is ablated.

In another embodiment, device 100C is navigated so that distal end 113 is in contact with at least a portion of AEN 101′, as depicted in FIG. 1F. An ablation mechanism is triggered to ablate at least a portion of AEN 101′. If desired the ablation can be repeated at the same location and/or the device repositioned and then triggered again at an adjacent location as needed so that all or substantially all of AEN 101′ on lateral nasal wall 100B is ablated.

In yet another embodiment, distal end 113 is navigated to NPN 102 on nasal septum 100A, as depicted schematically in FIG. 1G. An ablation mechanism is triggered, e.g., on the handle of the device 112, to activate the distal end 113 to ablate at least a portion of NPN 102. Similarly, this ablation step could be repeated until the desired portion of NPN 102 is ablated.

In yet another embodiment, distal end 113 is navigated to NPN 102 on nasal septum 100A, as depicted schematically in FIG. 1G. An ablation mechanism is triggered, e.g., on the handle of the device 112, to activate the distal end 113 to ablate at least a portion of NPN 102. Similarly, this ablation step could be repeated at the same location and/or the device repositioned and then triggered again at an adjacent location as needed until the desired portion of NPN 102 is ablated.

In yet another embodiment, distal end 113 is navigated to PEN (not shown). An ablation mechanism is triggered, e.g., on the handle of the device 112, to activate the distal end 113 to ablate at least a portion of PEN. Similarly, this ablation step could be repeated at the same location and/or the device repositioned and then triggered again at an adjacent location as needed until the desired portion of PEN is ablated.

FIG. 1H, shows the base of the nose 115, nostril 116, and cross section of device 100C. In one embodiment, device 100C is placed within the nostril so that distal end 113 is apposed on at least a portion of both AEN 101 and AEN 101′ so that both courses of AEN can be ablated at once.

In one embodiment, at least a portion of AEN 101 and AEN 101′ are ablated. In another embodiment, at least a portion of AEN 101 and NPN 102 are ablated. In another embodiment, at least a portion of AEN 101, AEN 101′, and NPN 102 are ablated. In another embodiment at least portion of AEN 101 and PEN is ablated. In another embodiment, at least a portion of AEN 101, AEN 101′, NPN 102, and/or PEN are ablated.

In one embodiment, device 100C is navigated through the nasal cavity. Distal end 113 is apposed on the mucosa of nasal septum 100A, an ablation mechanism is triggered, and at least a portion of nasal septal mucosa is ablated. The ablation step is repeated at the same location and/or the device repositioned and then triggered again at an adjacent location as needed until desired amount of mucosa of nasal septum 100A is ablated.

FIG. 2 shows nasal septum 200, anterior ethmoid artery 201, posterior ethmoid artery 202, sphenopalatine (nasopalatine) artery 203, greater palatine artery 204, superior labial artery 205, and Little's area (Kisserbach's plexus) 206. In one embodiment, device 100C is navigated through the nasal cavity. Distal end 113 is apposed on the mucosa overlaying Little's area 206, an ablation mechanism is triggered and all or substantially all the mucosa covering Little's area 206 is ablated.

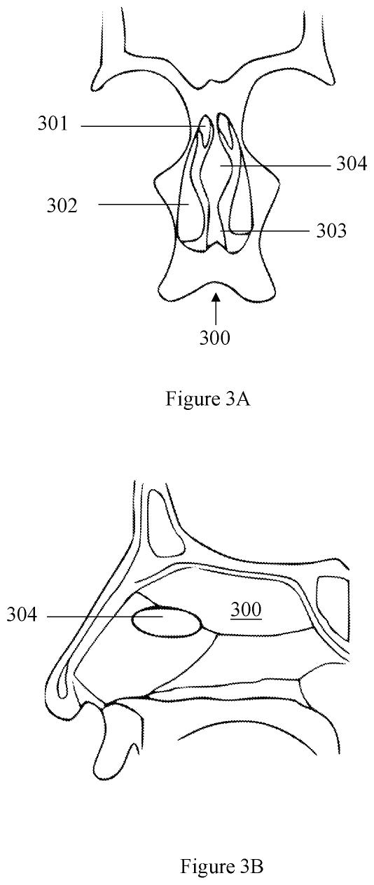

FIG. 3A shows the schematics of coronal cross section of nasal cavity 300, middle turbinate 301, inferior turbinate 302, nasal septum 303, and the approximate location of nasal swell body 304. FIG. 3B shows the schematics of the sagittal cross section of the nasal cavity 300 and the approximate location of nasal swell body 304. In one embodiment, device 100C is navigated through the nasal cavity 300 so that distal end 113 is apposed on the mucosa overlaying the nasal swell body 304, an ablation mechanism is triggered and all or substantially all the mucosa covering nasal swell body 304 is ablated.

In one embodiment, mucosa overlaying both Little's area 206 and nasal swell body 304 are ablated.

In one embodiment, the distal end 113 is designed to be atraumatic to the structures it encounters.

In another embodiment, device 100C has a cryogenic component. In other embodiments, device 100C uses radiofrequency, pulsed radiofrequency, laser, microwave, or other energy-based methods to effect ablation.

In one embodiment, device 100C is a cryoprobe using Joule's Thompson (JT) effect to create temperatures between −20° C. to −100° C. to effect nerve ablation. In this embodiment, a compressed refrigerant gas or liquid is ported through the cryoprobe via a small internal lumen to the proximity of the tip of the cryoprobe at which point the refrigerant gas or liquid exits the internal smaller lumen to enter a coaxial larger lumen. The expansion of the refrigerant gas or liquid during transition from the smaller lumen to the larger lumen cools distal end 113 to low temperatures.

In one embodiment, device 100C is a cryoprobe that uses nitrous oxide gas, liquid, or a mixture thereof, to create the extreme low temperatures. In another embodiment, the refrigerant gas or liquid may comprise carbon dioxide and in yet another embodiment, the refrigerant may comprise chlorofluorocarbon, hydrochlorofluorocarbon, hydrofluorocarbon or any mixtures thereof to produce the extreme, low temperatures.

In one embodiment, device 100C is a cryoprobe that uses liquid nitrogen to produce extreme low temperatures. In yet another embodiment, device 100C uses other methods than JT effect to produce low temperatures necessary for tissue ablation.

In another embodiment, device 100C is a cryoprobe that uses nitrous oxide gas, liquid, or a mixture thereof, to produce temperatures between −20° C. to −100° C. In one embodiment the flow of nitrous oxide gas or liquid is controlled in order to control the temperature of distal end 113. In another embodiment temperature of distal end 113 is adjusted in order to control the depth of freezing. For example, at lower temperatures, distal end 113 can freeze deeper into the target tissue compared to higher temperatures. The preferred depth for tissue freezing is about 100 μm to about 5 mm or more preferably between 100 μm to 3 mm.

In one embodiment, device 100C is a cryoprobe that uses nitrous oxide gas, liquid, or a mixture thereof, to produce temperatures between −20° C. to −100° C. at distal end 113. During ablation distal end 113 is apposed on the target tissue, the refrigerant gas is then allowed to flow through the cryoprobe by an open/close mechanism to begin ablation. Distal end 113 remains in contact with tissue for about, e.g., 10 seconds to about 120 seconds, or more preferably between, e.g., 10 seconds and 60 seconds, to freeze the target tissue. The flow of the refrigerant gas is then stopped, allowing the temperature of distal end 113 and the adjacent tissue to rise before distal end 113 is removed from the ablated tissue.

In another embodiment the dimensions of device 100C are such that it can operate within the confines of nasal cavity in conjunction with a visualization device such as nasal endoscope. For example, device 100C may have a shaft with a length ranging from, e.g., 4 cm to 12 cm and a diameter ranging from, e.g., 0.5 mm to 5 mm.

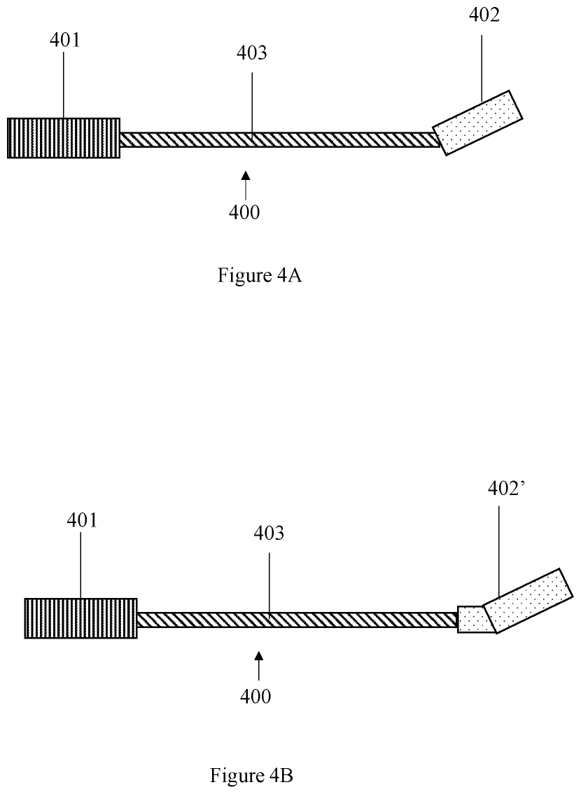

FIG. 4A shows the schematic of embodiment of device 400, proximal end 401, distal end 402, and elongated shaft 403. Distal end 402 sits at an angle relative to a longitudinal axis of shaft 403 making it easier for the device to contact the target tissue (as shown and described herein). The shaft 403 may accordingly include a proximal portion extending longitudinally and a distal portion which is angled relative to the proximal portion about an inflection point such as a curved or bent portion or pivoted portion where the angle may range from, e.g., 5 to 30 degrees. In one embodiment, all or part of shaft 403 is malleable allowing for adjusting the angle between distal end 402 and shaft 403. In another embodiment, elongated shaft 403 sits at an angle relative to proximal end 401 (not shown). FIG. 4B shows the schematic of embodiment of device 400, proximal end 401, distal end 402′, and elongated shaft 403. In this embodiment part or all of distal end 402′ is malleable making it easy for distal end 402′ to be bent either prior to or during the procedure to better appose on the target tissue.

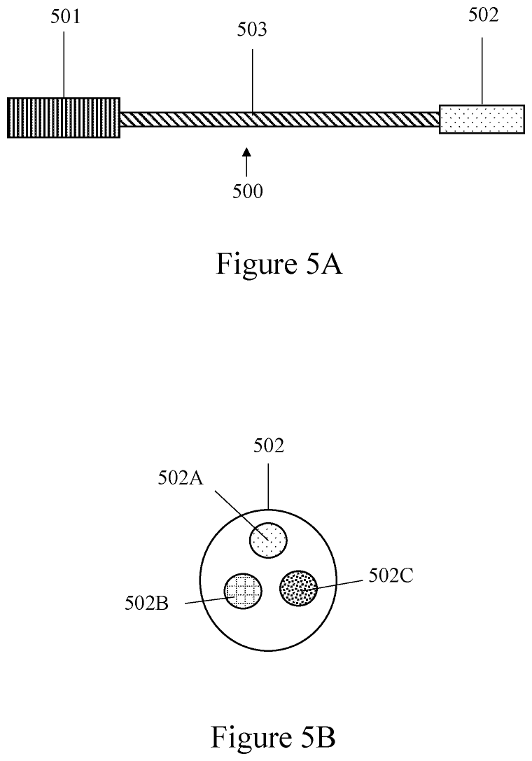

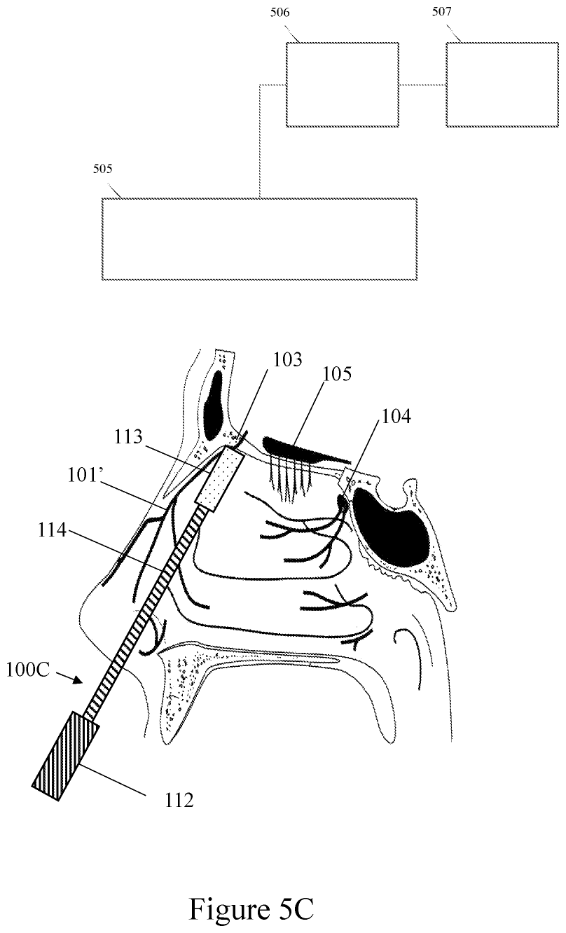

FIG. 5A shows device 500 with proximal end 501, distal end 502, and elongated shaft 503. FIG. 5B shows the cross section of distal end 502 where it comprises ablation effector 502A, illumination component 502B and imaging component 502C. This embodiment of device 500 makes it possible for device 500 to complete the ablation procedure without the need of an additional visualization instrument. In yet another embodiment device 500 includes a component (not shown) to facilitate its navigation using various visualization modalities for example facilitating its use with commercially available navigation systems such as the StealthStation™ ENT (Medtronic, Inc.).

An example is illustrated in FIG. 5C showing how a commercially available navigation system may be used to provide guidance in accurately positioning the distal end 113 of the device against a preselected tissue region or nerve fiber for treatment. The example shown illustrates how a camera or imaging system 505 may be positioned into proximity of the patient's head and nasal cavity. The imaging system 505 may be in communication with a controller 506 which may be under the control of the practitioner. A display and/or user interface 507 may also be available for displaying the images of the nasal cavity and its anatomical features obtained by the imaging system 505. The images may be displayed 507 as the device and distal end 113 are navigated within the nasal cavity allowing for the practitioner to guide the distal end 113 into proximity and/or against the preselected tissue region or nerve fiber for treatment.

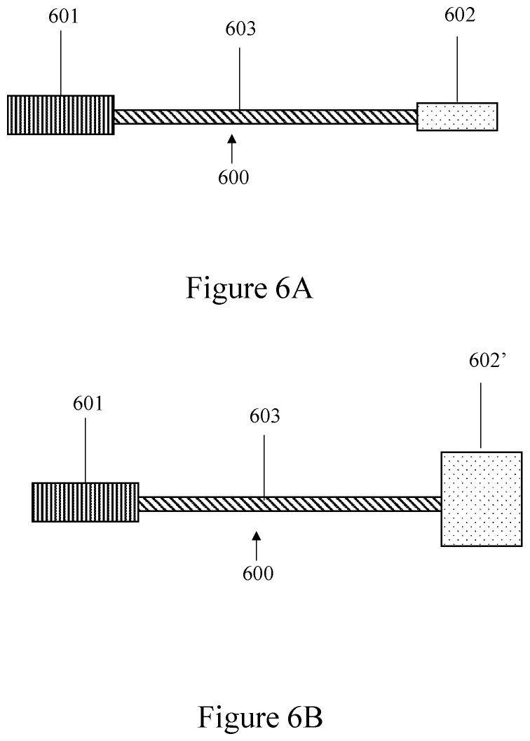

FIG. 6A depicts device 600 with proximal end 601, distal end 602, and elongated shaft 603. In one embodiment, distal end 602 can assume a collapsed state 602 and an expanded state 602′, as depicted in FIG. 6B. The collapsed state of distal end 602 makes navigation of device 600 through the nasal cavity easier, once in position, collapsed distal end 602 can be expanded to distal end 602′ in order to produce better apposition to tissue or to cover a larger surface area to be ablated.

In one embodiment device 600 has a cryogenic component that uses compressed gas as a refrigerant. In this embodiment the expansion of the refrigerant gas inside distal end 602 causes the cooling effect. In one embodiment the refrigerant gas used to cool cryogenic device 600 may be used to expand the distal end from configuration 602 to configuration 602′. Once the flow of the refrigerant gas is stopped, distal end 602′ warms to environmental temperatures and deflates to distal end 602 before device 600 is navigated out of the nasal cavity.

In another embodiment device 600 may use two or more different mechanisms to expand distal end 602. As an example, device 600 may use saline injection to expand distal end 602 to distal end 602′ and radiofrequency energy to ablate the target tissue.

In one embodiment the expandable component of distal end 602 is a balloon. In yet another embodiment the expandable component of distal end 602 is an expandable structure made of thermally conductive materials such as metals or shape memory alloys.

In another embodiment distal end 602 comprises a spray component (not shown) capable of spraying liquid or gas ablation material directly on the target tissue. In one configuration of this embodiment device 600 is a cryogenic device and distal end 602 sprays cryogenic material directly on target tissue to effect ablation. In another example device 600 sprays chemicals capable of inducing tissue ablation directly on the target tissue. Examples of such chemicals include ethanol, phenol, and zinc sulphate, etc. In yet another embodiment device 600 sprays therapeutic agents capable of ablating neurons, block their function, or otherwise interfere with their function. Example of such therapeutic agents include capsaicin and its analogues, including but not limited to Zucapsaicin, ALGRX-4975, Nonivamide, Resiniferatoxin, or combinations thereof, or sympatholytic agents such as alpha- and beta-adrenergic receptor antagonists (alpha blockers and beta blockers) as well as centrally acting agents such as clonidine, guanabenz, methyldopa, minoxidil, and reserpine.

In another embodiment distal end 602 is an injection needle capable of injecting ablative liquid chemicals or therapeutic agents directly into the target tissue. Such liquid chemicals or therapeutic agents are capable of effecting ablation once injected. Examples of chemicals used for ablation include ethanol, phenol, zinc sulphate and examples of therapeutic agents include capsaicin and its analogues.

In any of the embodiments described, various features of the device between the different embodiments may be combined in any number of combinations. Hence, any of the devices may be used to effect the treatment of any of the nerve bodies described herein to effect the treatment of a particular disease.

In one embodiment the mucosa of nasal septum as depicted in FIG. 1A, is stimulated in order to increase cerebral blood flow. In one embodiment, nasal septal mucosa is chemically stimulated. In another embodiment, the septal mucosa is mechanically stimulated. In yet another embodiment, the septal mucosa is thermally stimulated. In one embodiment, septal mucosa is electrically stimulated. In another embodiment the mucosa is stimulated by light.

In another embodiment stimulating septal mucosa includes stimulating mucosa overlaying the Little's area as depicted in FIG. 2.

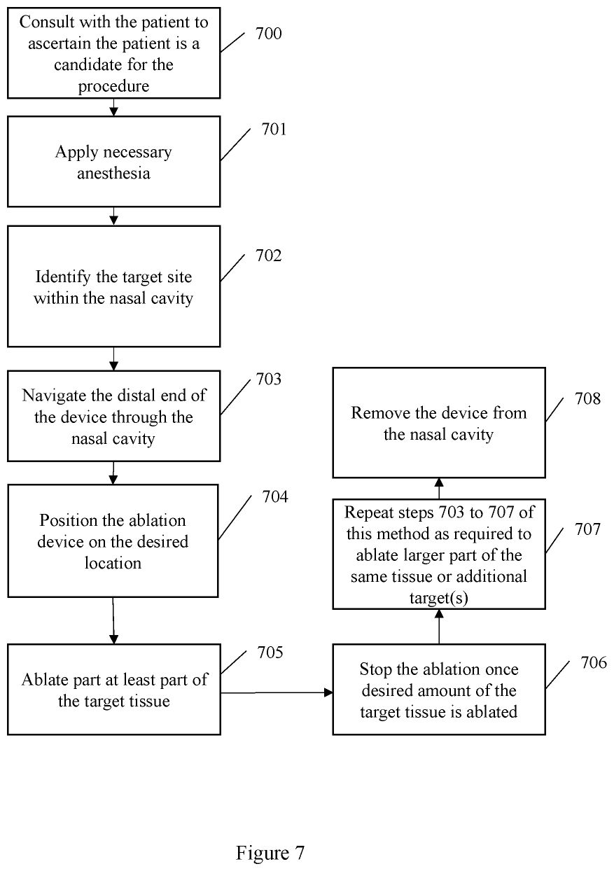

Step by step of one method prescribed for treating diseases is depicted in the flow diagram of FIG. 7:
a. Consult with the patient and ascertain that the patient is a candidate for the current procedure 700,
b. Apply necessary anesthesia to the region of tissue to be treated 701,
c. Identify the target tissue within the nasal cavity 702,
d. Navigate the distal end of an ablation device into the nasal opening and through the nasal cavity 703,
e. Position the ablation device on or in proximity to the desired location 704,
f. Ablate part at least part of the target tissue 705,
g. Stop the ablation once desired amount of the target tissue is ablated 706,
h. Repeat steps 703 to 707 of this method as required to ablate larger part of the same target or additional target(s) 707,
i. Remove the device from the nasal cavity 708.

With respect to the identification of the appropriate target tissue, one or more of the various nerves may be targeted depending upon the condition to be treated. Examples are described in further detail below.

In the method described above, some of the individual steps may be omitted entirely or applied in an alternative order.

In one embodiment of the method of this invention diseases to be treated include but are not limited to nasal hyperreactivity, rhinitis, rhinosinusitis, headaches including migraine, and olfactory dysfunction.

In one embodiment of the method contemplated, the target tissue is all or substantially all the nerve fibers passing through anterior ethmoid foramen. In another embodiment, the target tissue is all or substantially all the nerve fibers passing through anterior and posterior ethmoid foramina. In another embodiment, the target tissue is all or substantially all the nerve fibers passing through anterior ethmoid and sphenopalatine foramina. In another embodiment, the target tissue is all or substantially all the nerve fibers passing through anterior and posterior ethmoid, and sphenopalatine foramina.

In yet another embodiment the target tissue is AEN coursing over nasal septum. In another embodiment the target tissue is AEN coursing over nasal septum and its branch coursing over lateral nasal wall. In another embodiment the target tissue is AEN and NPN on the nasal septum. In another embodiment the target tissue is AEN and NPN on the nasal septum and the AEN branch coursing over lateral nasal wall.

In one embodiment of the method the target tissue is at least part of the mucosa of nasal septum. In another embodiment the target tissue includes mucosa over the little's are. In another embodiment target tissue includes mucosa over the mid septum or nasal swell body. In another embodiment target tissue includes mucosa over anterior aspect of inferior turbinate and/or middle turbinate.

In one embodiment of the method described herein, the target tissue is the anterior aspect of the nasal septum which can be accessed without the need for endoscopic visualization.

In one embodiment of the method described herein, the step to ascertain the appropriateness of this therapy includes applying local anesthetics to the target tissue in order to observe its impact on the disease state, prior to ablation procedure.

In one embodiment of the method described herein, the step to identify the target nerve or nerves can include using a nerve tissue visualization aid (e.g., dyes) or other techniques to distinguish nerve tissue from surrounding tissues. In another embodiment, the step to identify target nerve includes methods of nerve stimulation (e.g. pressurization of a sinus cavity) and/or nerve monitoring techniques commonly known in the art.

In one embodiment of the method contemplated in this invention, the ablation device uses energy to ablate.

In one embodiment of this method, the ablation device is a cryoprobe that uses compressed nitrous oxide gas, liquid, or a mixture thereof, to create temperatures in the range of −20° C. to −100° C. to effect cryoablation. In one embodiment the preferred cryoprobe temperature is between −20° C. to −60° C.

In one embodiment of this method, the ablation device is placed is a cryoprobe. Once the target tissue is identified, the cryoprobe is placed against the target tissue and an ablation mechanism is triggered to freeze the target tissue to temperatures in the range of −20° C. to −100° C. In one embodiment the mechanism to trigger ablation is to control the flow of the cryogenic gas into the cryoprobe. Triggering ablation starts the flow of the cryogen, bringing the temperature of the probe to desired low temperature. In one embodiment, the ablation is conducted for between 10 seconds and 120 second. In a preferred scenario the ablation is done for between 10 seconds and 60 seconds. Once the ablation is complete, the flow of cryogen gas into the cryoprobe is stopped. The cryoprobe remains on the tissue while the tissue warms back to environmental temperatures before the cryoprobe is removed or relocated to a new tissue target location for a subsequent ablation.

In another embodiment the ablation is effected using chemical or therapeutic agents.

It is herein contemplated that other medical therapies such as use of pharmaceutical agents can be conducted in combination with the methods described herein in order to improve the clinical outcomes of the patient.

As described herein, one or more of the various nerves and/or tissues may be treated (e.g., ablation and/or stimulation) depending upon the condition to be mitigated. Examples of various conditions and the respective nerves and/or tissues to be treated are described below in Table 1:

TABLE 1
Conditions for treatment.

| | | Treatment |
|---|---|---|
| Condition | Nerve(s) To Be Treated | Modality |
| Headache, including but not | AEN (one or both septal | Ablation |
| limited to migraine headaches | and lateral courses) | |
| and neurological diseases | and/or NPN and/or PEN | |
| attributed to upper airway | and/or at least a | |
| viral/bacterial infection | portion of nasal septum | |
| such as Covid 19 | mucosa | |
| Nasal hyperreactivity, | AEN (one or both septal | Ablation |
| including but not | and lateral courses) and/ | |
| limited to rhinitis | or NPN and/or PEN and/or | |
| and rhinosinusitis | at least a portion of | |
| | nasal septum mucosa | |
| Olfactory dysfunction | AEN (one or both septal | Ablation |
| including but not limited | and lateral courses) | |
| to dysfunction those | and/or PEN | |
| attributed to upper airway | | |
| viral/bacterial infection | | |
| such as Covid 19 | | |
| Cerebrovascular or | At least a portion of | Stimulation |
| neurodegenerative disease | nasal septum mucosa | |

Moreover, Table 1 illustrates examples of various conditions, the targeted nerves and/or tissues to the treated (e.g., ablated and/or stimulated). Any of the various devices and methods as described herein may be used to provide the treatment (e.g., ablation and/or stimulation), as practicable, and any of the various features between different embodiments are intended to be used in various combinations to effect a treatment.

In one method of the current application, the patient is diagnosed with a headache disorder by the physician and a candidate for current procedure. The device used in the procedure is a cryoprobe with a malleable distal end, capable to reach temperatures between −20° C. to −100° C., using nitrous oxide as cryogen and the Joules Thompson effect. The surgeon uses an endoscopic visualization with or without the help of a navigation instrument to identify the course of AEN and/or NPN. The cryoprobe is then navigated through the nasal cavity to reach the desired part of the nerve to be ablated. The distal end of the cryoprobe is placed on the nerve and the ablation mechanism is triggered by allowing the cryogen to flow though the cryoprobe. The nerve is then frozen for a period of about 10 seconds to 60 seconds. The flow of cryogen is stopped, and the distal end is allowed to thaw. Once the distal end is released from tissue, the ablation procedure might be repeated on additional locations until desired length of nerve(s) is ablated. Once the ablation is complete, the cryoprobe is navigated out of the nasal cavity.

In one method of the current application, the patient is diagnosed with nasal hyperreactivity by the physician and a candidate for current procedure. The device used in the procedure is a cryoprobe with a malleable distal end, capable to reach temperatures between −20° C. to −100° C., using nitrous oxide as cryogen and the Joules Thompson effect. The surgeon uses an endoscopic visualization with or without the help of a navigation instrument to identify at least portion of nasal septal mucosa to be ablated. The cryoprobe is then navigated through the nasal cavity to reach the desired part of the nasal septum. The distal end of the cryoprobe is placed on the tissue and the ablation mechanism is triggered by allowing the cryogen to flow though the cryoprobe. The mucosa is then frozen for a period of about 10 seconds to 60 seconds. The flow of cryogen is stopped, and the distal end is allowed to thaw. Once the distal end is released from tissue, the ablation procedure might be repeated on additional locations until desired surface area of the nasal septum mucosa is ablated. Once the ablation is complete, the cryoprobe is navigated out of the nasal cavity.

The applications of the devices and methods discussed above are not limited to the treatments described but may include any number of further treatment applications. Moreover, such devices and methods may be applied to other treatment sites within the body. Modification of the above-described assemblies and methods for carrying out the invention, combinations between different variations as practicable, and variations of aspects of the invention that are obvious to those of skill in the art are intended to be within the scope of the claims.

1. A method for treating dysfunctions or diseases in a patient, comprising:
introducing a treatment device into a nasal cavity of a patient, the treatment device having a proximal end, a distal end, an elongate shaft therebetween, and an ablation effector disposed at or near the distal end of the device;
navigating the distal end into proximity of nerve fibers passing through an anterior ethmoid foramen or a posterior ethmoid foramen; and
ablating all or substantially all of the nerve fibers at the anterior ethmoid foramen or at the posterior ethmoid foramen such that the dysfunction or disease is mitigated.

2. The method of claim 1, wherein the dysfunctions or diseases to be treated comprise nasal hyperreactivity, rhinitis, nasal obstruction, post-nasal drip, headache, high cerebral blood flow, trigeminal hyperreactivity, trigeminal dysfunction, upper respiratory viral infection, olfactory dysfunction, or pain associated with nasal contact points.

3. The method of claim 1, wherein ablating comprises ablating all or substantially all of the nerve fibers via cryoablation, radiofrequency, pulsed radiofrequency, microwave, ultrasonic, thermal energy, mechanical force, or applying at least one chemical agent or at least one therapeutic agent to the nerve fibers.

4. The method of claim 3 wherein the at least one chemical agent or at least one therapeutic agent is selected from the group consisting of ethanol, phenol, capsaicin, and capsaicin analogues.

5. The method of claim 1, wherein ablating all or substantially all of the nerve fibers comprises applying a cryoprobe at temperatures between −20° to −100° C. to effect ablation.

6. The method of claim 5, wherein ablation is effected for about 10 seconds to about 120 seconds to effect ablation.

7. The method of claim 5, wherein ablating all or substantially all of the nerve fibers comprises ablating to a depth of 100 μm to 3 mm in the tissue.

8. The method of claim 1, wherein navigating the distal end comprises imaging the nasal cavity via a navigation aid while advancing the distal end of the treatment device within the nasal cavity prior to ablating all or substantially all of the nerve fibers.

9. A method for treating dysfunctions or diseases in a patient, comprising:
introducing a treatment device into a nasal cavity of a patient, the treatment device having a proximal end, a distal end, an elongate shaft therebetween, and an ablation effector disposed at or near the distal end of the device;
navigating the distal end to within a 5 mm radius of an arch where a middle turbinate inserts superiorly to a lateral nasal wall; and
ablating a portion of a mucosa within the radius and at least one nerve covered by the mucosa such that the dysfunction or disease is mitigated.

10. The method of claim 9, wherein the dysfunctions or diseases to be treated comprise nasal hyperreactivity, rhinitis, nasal obstruction, post-nasal drip, headache, high cerebral blood flow, trigeminal hyperreactivity, trigeminal dysfunction, upper respiratory viral infection, olfactory dysfunction, or pain associated with nasal contact points.

11. The method of claim 9, wherein ablating comprises ablating all or substantially all of the nerve fibers via cryoablation, radiofrequency, pulsed radiofrequency, microwave, ultrasonic, thermal energy, mechanical force, or applying at least one chemical agent or at least one therapeutic agent to the nerve fibers.

12. The method of claim 11 wherein the at least one chemical agent or at least one therapeutic agent is selected from the group consisting of ethanol, phenol, capsaicin, and capsaicin analogues.

13. The method of claim 9, wherein ablating all or substantially all of the nerve fibers comprises applying a cryoprobe at temperatures between −20° to −100° C. to effect ablation.

14. The method of claim 13, wherein ablation is effected for about 10 seconds to about 120 seconds to effect ablation.

15. The method of claim 13, wherein ablation is effected to a depth of 100 μm to 3 mm in the tissue.

16. The method of claim 9, wherein navigating the distal end comprises imaging the nasal cavity via a navigation aid while advancing the distal end of the treatment device within the nasal cavity prior to ablating the portion of the one nerve.

17. A method for treating dysfunctions or diseases in a patient, comprising:
introducing a treatment device into a nasal cavity of a patient, the treatment device having a proximal end, a distal end, an elongate shaft therebetween, and an ablation effector disposed at or near the distal end of the device;
navigating the distal end into proximity of at least a portion of mucosa of the nasal septum overlaying a vascular plexus; and
ablating the portion of the mucosa such that the dysfunction or disease is mitigated.

18. (canceled)

19. The method of claim 17, wherein the dysfunctions or diseases to be treated comprise nasal hyperreactivity, rhinitis, nasal obstruction, post-nasal drip, headache, high cerebral blood flow, trigeminal hyperreactivity, trigeminal dysfunction, upper respiratory viral infection, olfactory dysfunction, or pain associated with nasal contact points.

20. The method of claim 17, wherein ablating comprises ablating the portion via cryoablation, radiofrequency, pulsed radiofrequency, microwave, ultrasonic, thermal energy, mechanical force, or applying at least one chemical agent or at least one therapeutic agent to the mucosa.

21. The method of claim 20, wherein the at least one chemical agent or at least one therapeutic agent is selected from the group consisting of ethanol, phenol, capsaicin, and capsaicin analogues.

22. The method of claim 17, wherein navigating the distal end comprises imaging the nasal cavity via a navigation aid while advancing the distal end of the treatment device within the nasal cavity prior to ablating the portion of the mucosa.

23. A method for increasing cerebral blood flow, comprising treating at least part of a nasal septal mucosa.

24. The method of claim 23, wherein treating at least part of the nasal septal mucosa comprises stimulating the nasal septal mucosa.

25. The method of claim 23, wherein treating the nasal septum mucosa comprises stimulating a portion of the nasal septum mucosa covering Little's area.

26. The method of claim 23, wherein treating the nasal septal mucosa comprises applying at least one modality of mechanical, chemical, electrical, or light stimulation.

27. The method of claim 23, wherein treating the nasal septal mucosa comprises increasing cerebral blood flow such that symptoms related to cerebrovascular or neurodegenerative diseases are mitigated.

28. A method for treating headache in a patient, comprising:
introducing a treatment device into a nasal cavity of a patient, the treatment device having a proximal end, a distal end, an elongate shaft therebetween, and an ablation effector disposed at or near the distal end of the device;
navigating the distal end into proximity of an anterior ethmoid foramen including at least an anterior ethmoid nerve which passes through the anterior ethmoid foramen and innervates a region of tissue; and
ablating all or substantially all of the anterior ethmoid nerve at the anterior ethmoid foramen such that the headache is mitigated.

29. The method of claim 28, wherein ablating comprises ablating all or substantially all of the nerve fibers via cryoablation, radiofrequency, pulsed radiofrequency, microwave, ultrasonic, thermal energy, mechanical force, or applying at least one chemical agent or at least one therapeutic agent to the nerve fibers.

30. The method of claim 9 further comprising ablating a second portion of a second mucosa at a second location within 5 mm opposite to the arch where the middle turbinate inserts superiorly to the lateral nasal wall.

31. The method of claim 9 wherein the method for treating dysfunctions or diseases comprises a method for treating headache in the patient such that the headache is mitigated.
